(19) United States
(12) Patent Application Publication
(10) Pub. No.: US 2008/0060659 A1
(43) Pub. Date: Mar. 13, 2008

(54) Adjustable dental apparatus for reducing snoring and improving sleep disordered breathing

(75) Inventors: Richard A. Bonato, Carp (CA); Donald C. Bradley, Kanata (CA)

Correspondence Address: PERLEY-ROBERTSON, HILL & MCDOUGALL LLP, 1400-340 Albert Street, OTTAWA, ON, K1R 0A5, US

(73) Assignee: Braebon Medical Corporation

(21) Appl. No.: 11/519,871

(22) Filed: Sep. 13, 2006

(51) Int. Cl. A61F 5/56

(52) U.S. Cl. 128/848

(57) ABSTRACT

A two part therapeutic intra-oral mandibular appliance is provided with single adjustment means facilitating mandibular protrusion or retrusion to a desired position while the appliance is being worn. To this end there is provided separate upper and lower U-shaped arches, each having a front section, two side sections, a top surface and bottom surface. The top surface of the upper arch and the bottom surface of the lower arch are respectively configured to receivingly engage at least some of a user's upper and lower teeth. The bottom surface of at least an anterior portion of the front section of the upper arch is recessed along a transverse step relative to the remainder of the bottom surface of the upper arch. A post is centrally located in the front section of the lower arch and is designed to extend upwardly from the top surface of the lower arch into the recessed front section of the upper arch. The adjustment means for selectively varying the distance between the post and the step and which results in the advancement or retraction of a wearer's mandible, comprises an elongate rod one end of which is threadedly interconnected to a bore extending through the post, and the other end of which abuts the step. The rod can be accessed for rotation while the appliance is being worn by inserting drive means such as a screwdriver into the bore so as to drivingly engage the one end of the rod threadedly connected to the post.

### FIELD OF INVENTION

This invention relates to therapeutic intraoral mandibular appliances or devices that reposition the mandible and, in particular, to an anti-snoring apparatus which also improves sleep disordered breathing.

### BACKGROUND OF INVENTION

Approximately 50% of the general population suffers from snoring with about 3-5% of the population suffering from a more severe form of sleep disordered breathing called obstructive sleep apnea, a phenomenon whereby the sleeper stops breathing during sleep due to a narrowing or occlusion of the pharyngeal airway during sleep. The use of oral appliances for the treatment of snoring and sleep disordered breathing has been well documented in scientific literature and are effective because they advance the lower jaw and maintain the mandible in a preferred anterior position thereby improving airway patency

### DESCRIPTION OF RELATED ART

Known appliances fall into either one-piece or two-piece designs, and all of which advance the mandible using various techniques. Cook (U.S. Pat. No. 5,003,994), Hays et al. (U.S. Pat No. 5,092,346), Shapiro et al. (U.S. Pat. No. 5,117,816), and Hays (U.S. Pat. No. 5,277,202) are representative of one-piece designs which advance the mandible to open the airway passage reducing or eliminating snoring and improving breathing during sleep. Toone (U.S. Pat. No. 4,901,737), Halstrom (U.S. Pat. Nos. 5,365,945 & 6,729,335), Thornton (U.S. Pat. Nos. 5,427,117, 6,305,376 & 6,516,805), Hilsen (U.S. Pat Nos. 5,611,355 & 5,884,628), Kidd (U.S. Pat. No. 5,829,441), Orrico (U.S. Pat. No. 6,170,485), and Gaskell (U.S. Pat No. 6,845,774) are examples of various types of two-piece adjustable mandibular advancement appliances

Of the various two-piece designs into which category the subject invention falls, the most notable prior art includes the two piece anti-snoring adjustable intra-oral appliance disclosed by Toone in U.S. Pat. No. 4,901,737 and which permits mandibular protrusion or retrusion employing for that purpose two laterally spaced apart rods which are threadedly connected to the side sections of the lower part of the two-part appliance, with the free ends of the two rods abutting shoulders which are provided in the side sections of the upper part of the appliance. Forward or rearward relative positioning of the lower portion to the upper portion of the appliance is achieved by advancing or retracting the two threaded rods at their two side locations.

A medical oral appliance sold under the trademark SOMNOGUARD® and available from Tomed Dr. Toussaint GmbH Lindberger Strauss, 3ad-64625 Bensheim, Germany concerns another type of two-piece adjustable intra-oral appliance. The bottom surface of the upper piece or arch of the appliance has attached to it a laterally extending C-shaped slot or channel, with the elongate opening in the channel facing forward when worn. The bottom section or arch, on its upper surface, is provided with a centrally located post which threadingly receives on the interior side of the post a threaded rod. The threaded rod itself, at its free end, is provided with an enlarged head which is dimensioned so as to be slidingly received within the c-shaped slot of the upper section. Forward and rearward adjustment of the lower section relative to the upper section is achieved by separating the two sections by slidingly disengaging the head of the rod from the C-shaped slot, and then advancing or retracting the threaded rod within the post by turning the head of the rod.

For adjustment purposes, it is apparent that Toone's arrangement as described above requires left and right side adjustment which can result in misalignment and wearer discomfort. The SOMNOGUARD® appliance, on the other hand, is not capable of adjustment at the time it is being worn since the rotatable head of the threaded rod cannot be accessed unless the joined together upper and lower arches are first removed from the oral cavity. Further, due to the sliding interconnection of the upper arch to the lower arch, the inability of the two pieces to be readily separated when being worn can cause distress or anxiety to the person wearing the appliance if the removal of the appliance, such as when coughing, is urgently required.

### SUMMARY OF THE INVENTION

In accordance with the present invention, there is provided a novel two-piece therapeutic intra-oral mandibular advancement appliance which is readily adjustable when being worn and which, if need be, can be removed from a wearer's mouth quickly either as one unit or as separate pieces.

In keeping with the foregoing, there is provided separate upper and lower U-shaped arches, each having a front section, two side sections, a top surface and a bottom surface. The arches themselves can be formed or cast by a dental professional using Alginate and accompanying materials normally used in the preparation of custom dental appliances or may by formed by employing for that purpose suitable thermodeformable material such as Elvax® so that the top surface of the upper arch and the bottom surface of the lower arch are configured to receivingly engage at least some of user's upper and lower teeth in a manner which is well known in the art.

The bottom surface of at least an anterior portion of the front section of the upper arch is recessed along a transverse shoulder or step relative to the remainder of the bottom surface of the upper arch. A post is centrally located in the front section of the lower arch and is designed to extend upwardly from the top surface of the lower arch into the recessed front section of the upper arch.

In accordance with my invention, and in order to selectively vary the distance between the post and the step which necessarily results in mandibular protrusion or retrusion when the appliance is being worn, screw adjustment means is provided for and which comprises an axially adjustable rod, one end of which is screw threaded to the post. The other, free end of the rod, abuts the transverse step or shoulder. As the end of the rod which is screw threaded to the post is accessible from the anterior side of the post, employing suitable screw drive means such as a screwdriver or hex key, the rod can be threadedly rotated, and thus the distance between the post and step adjusted while the appliance is located in situ in the oral cavity. In order to reduce friction between the step or shoulder and the free end of the rod which bears against the free end of the rod can be topped with any suitable friction reducing plastic material, such as Teflon®.

While the threaded rod can directly engage complimentary threads provided in a bore extending through the post, in accordance with an alternative embodiment to my invention, a screw cage can advantageously be embedded within the post. The rod is threadedly attached to one end of the cage with the rod and extending interior of the cage being provided with an enlarged screw head which is captivated within the cage. An opening, which is smaller than the screw head, is provided at the other end of the cage and through which a screwdriver or like can be inserted for engagement with the screw head, thereby enabling the rod to be rotated. By employing a screw cage, the rod cannot be inadvertently separated from the post during adjustment.

From the foregoing, it will be apparent that the screw adjustment means permits both lateral and vertical movement of the mandible and also lingual freedom, since the shoulder or step of the upper arch is preferably located anterior to the tongue of the wearer or at least forward of the transverse location of a user's maxillary molars.

Since there does not exist any mechanical attachment between the upper and lower arches and which is characteristic of some two-part appliances, the single screw adjustment means as described herein decreases the amount of hardware in the mouth where mechanical attachments such as hooks, clasps, screws, sliders and the like are employed, thus occupying less space in the mouth and thereby improving patient comfort, compliance and efficacy. Further, the appliance of my invention does not require the need of any special jaw movements or motions to engage or disengage the device, and allows for improved ease of use, as well as improved ability to talk or drink. However, should it be desired to control, within limits, the permitted amount of lateral or up and down movement, there can advantageously be provided in the shoulder or step, a centrally located and transversely extending slot for receiving the free end of the rod. It will be appreciated that the depth of the slot will dictate the degree of forward mandibular movement required to disengage the rod from the slot, and hence the upper and lower sections from one another.

Although the plane of the transverse step or shoulder, when the appliance is being worn, is generally vertical, in an alternative embodiment to the above-described slot arrangement, and if desired, the plane can be made upwardly inclined in a direction away from the front section of the upper arch. The contact between the free end of the adjustment rod carried by the post on the lower arch and the inclined shoulder or step of the upper arch has the effect of biasing the two arches together when being worn and as a consequence, assist in reducing inadvertent mandibular retrusion during sleep.

### BRIEF DESCRIPTION OF THE SEVERAL VIEWS OF THE DRAWINGS

In the drawings, which illustrate preferred embodiments of the invention:

FIG. 1 is a side view of the two-piece appliance;

FIG. 2 is a bottom plan view of the upper U-shaped arch;

FIG. 3 is a rear view of the arch shown in FIG. 2;

FIG. 4 is a front view of the upper arch of FIGS. 2 and 3 and which additionally, illustrates the optional transverse slot in the step of the upper arch;

FIG. 5 is a top plan view of the lower U-shaped arch;

FIG. 6 is a front elevational view of the lower arch seen in FIG. 5;

FIG. 7 is a similar side view to that of FIG. 1, but wherein the transverse step or shoulder of the upper arch is upwardly inclined away from the front portion of the arch; and

FIG. 8 is an enlarged cut away and sectional view of the post of the lower arch which has embedded in it a screw cage which captivates the adjustment rod.

### DETAILED DESCRIPTION OF THE DRAWINGS

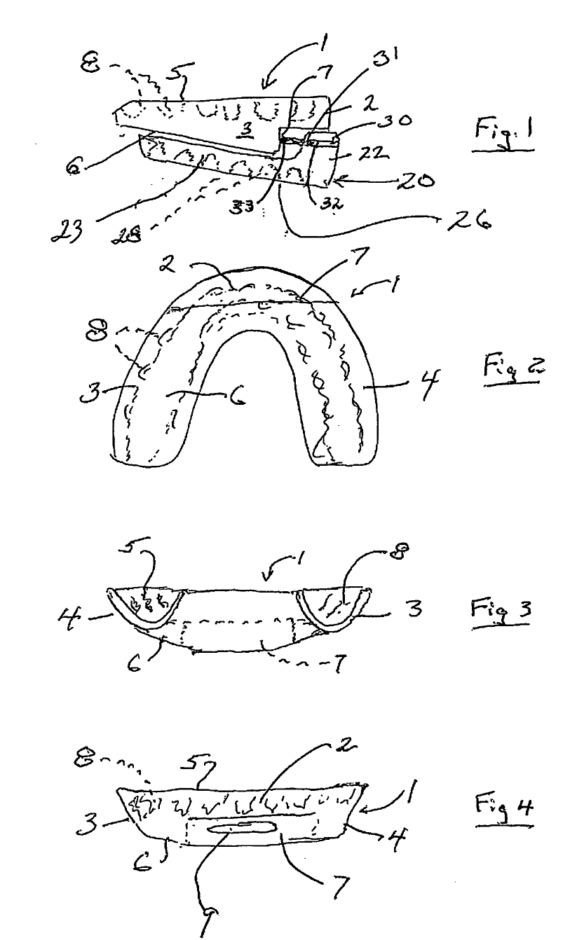

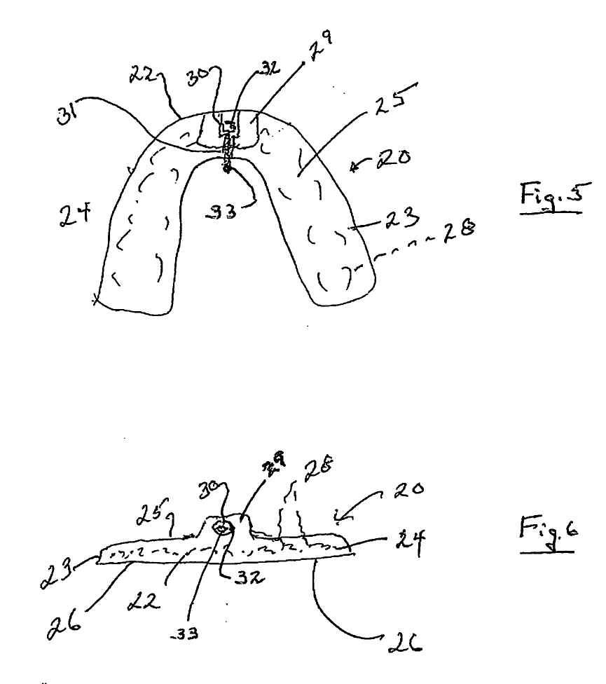

The two-piece intra-oral mandibular advancement appliance as illustrated in FIG. 1 comprises upper arch 1 and which seen from FIGS. 2, 3 and 4, includes a front section 2, two side sections 3 and 4, a top surface 5 and a bottom surface 6.

The bottom surface 6 of at least an anterior portion of front section 2 of upper arch 1 is recessed along a transverse step or shoulder 7 best seen in FIGS. 1, 2 and 4. Dental imprints of the dentures of the person wearing the appliance (not shown) are illustrated at 8 in the top surface 5 of upper arch 1.

The lower U-shaped arch 20 illustrated in FIGS. 1, 5 and 6, like upper arch 1, consists of a front section 22, two side sections 23 and 24, a top surface 25 and a bottom surface 26. Imprints of the wearer's lower dentures located in the lower surface of arch 20 are illustrated at 28.

A characterizing feature of lower arch 20 is post 29 which is located centrally of front section 22 and which projects upwardly from the top surface 25 of arch 20. Aperture or bore 30 extends through the post as illustrated. Screw or threaded rod 31 having enlarged screw head 32 is attached to post 29 by being threadedly interconnected to bore 30 in post 29.

As best seen in FIG. 1, the free end of rod 31 extends into the recess formed in the bottom surface 6 of arch 2 and abuts shoulder 7. By inserting a screwdriver, hex key or the like (not shown) into the anterior portion of bore 30 of the appliance when it is being worn, rod 31 can be rotated in a known manner in order to alter the distance between shoulder or step 7 and post 29 and thus cause a corresponding linear advancement or retraction of the wearer's mandible, without any lateral misalignment. In order to reduce friction between the free end of the rod 31 and shoulder 7 against which it abuts, the free end can be tipped with any suitable friction reducing plastic material as seen at 33 in FIGS. 1 and 5.

With reference to FIG. 4, it will be seen that step or shoulder 7 forming the recess in bottom surface 6 of upper arch 2 is provided with elongate slot 9 which is dimensioned to receive therein the free end of threaded rod 31 extending from post 29 as best seen in FIG. 1. In situations where the appliance of this invention is provided with an elongate slot 9 seen in FIG. 4, when engaged by rod 31 carried by the lower arch, both lateral and vertical movement of the mandible relative to upper arch 1 is constrained by the dimensioning of slot 9, yet allowing the upper and lower arches to be readily separated one from the other upon limited advancement of the mandible which carries with it lower arch 20. This slot is an advantageous additional feature to my invention as it enables control over the amount of vertical and horizontal movement and reduces the likelihood of inadvertent mandibular retrusion during sleep.

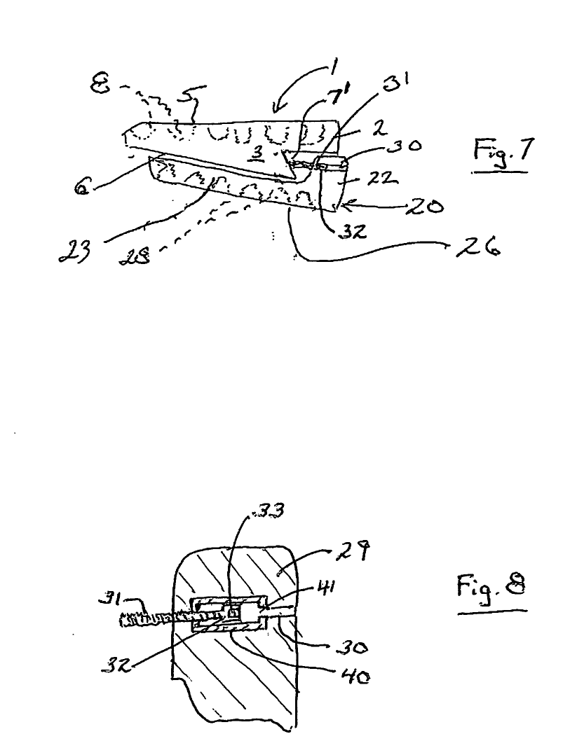

As seen in FIG. 7, transverse shoulder or step 7¹ is upwardly inclined away from front section 2. Adjustable rod 31 carried by post 29 of lower arch 20 abuts step 7¹. Due to the inclined plane of step 7¹, lower arch 20 when being worn is biased in an upward direction towards upper arch 3 thus serving to reduce inadvertent mandibular retrusion.

As seen in FIG. 8, if desired, post 29 can be provided with a screw cage 40 which at one of its ends is threadedly attached to adjustment screw or rod 31 as illustrated. Rod 31 at its end interior of the cage terminates at enlarged screw head 32 which itself is provided with a slot or socket enabling rotation of adjustment rod 31 when a screwdriver, hex key or the like (not shown) is inserted through bore 30 on the anterior side of the post and through the opening 41 provided in the cage for the purpose of positively engaging screw head 32 so that rod 31 can be rotated for adjustment purposes. As head 32 is larger than opening 41, actual displacement of adjustment rod 31 is limited to the location of captivated screw head 32 within the cage. This alternative form of construction ensures that the adjustable rod 31 cannot be separated from post 29 to which it is attached while permitting the free end of the rod to be extended and retracted relative to the post to which it is attached.

We claim:

1. A therapeutic intra-oral mandibular advancement appliance comprising:
(a) separate upper and lower U-shaped arches, each having a front section, two side sections, a top surface and a bottom surface;
(b) the top surface of the upper arch and the bottom surface of the lower arch being respectively configured to receivingly engage at least some of a user's upper and lower teeth;
(c) the bottom surface of at least an anterior portion of said front section of said upper arch being recessed relative to the remainder of the bottom surface of said upper arch along a transverse step;
(d) a post centrally located in the front section of said lower arch and which is extendable upwardly from the top surface of the lower arch into said recessed front section of said upper arch; and
(e) screw adjustment means for selectively varying the distance between said post and said step comprising an axially adjustable rod, a first end of which is screw threaded to said post and rotatable within said post from said first end.

2. The appliance as claimed in claim 1, wherein said remainder of the bottom surface of said upper arch is engageable with the top surface of said lower arch, and the bottom surface of said anterior portion of said front section of said upper arch is recessed relative to the top surface of the front section of said lower arch.

3. The appliance as claimed in claim 1, wherein said post is integral with said lower arch.

4. The appliance as claimed in claim 1, wherein said transverse step is positioned forward of a transverse location of said user's maxillary molars.

5. The appliance as claimed in claim 1, wherein said rod is threadedly interconnected to a bore extending through said post.

6. The appliance as claimed in claim 1, wherein said screw adjustment means further includes a screw cage embedded in said post and said rod at its said first end terminates at an enlarged screw head which is captured in said screw cage.

7. The appliance as claimed in claim 1, wherein said transverse step includes a centrally located and transversally extending slot for receiving a second end of said rod.

8. The appliance as claimed in claim 1, wherein said transverse step is upwardly inclined in a direction away from said front section of said upper arch.

9. The appliance as claimed in claim 5, wherein a second end of said rod which abuts said step when the appliance is being worn is tipped with a friction reducing plastic material.
